(19) United States
(12) Patent Application Publication
(10) Pub. No.: US 2019/0110947 A1
(43) Pub. Date: Apr. 18, 2019

(54) THERAPEUTIC ROLLER DEVICE

(71) Applicants: George Mbella Ekema, Akron, OH (US); Marjet Danteel HEITZER, Akron, OH (US)

(72) Inventors: George Mbella Ekema, Akron, OH (US); Marjet Danteel HEITZER, Akron, OH (US)

(21) Appl. No.: 16/094,608

(22) PCT Filed: Apr. 24, 2017
(86) PCT No.: PCT/US17/29050

§ 371 Date: Oct. 18, 2018

Related U.S. Application Data
(60) Provisional application No. 62/328,335, filed on Apr. 27, 2016.

(51) Int. Cl. A61H 15/02; A61H 15/00; A61F 7/10; A61F 7/02

(52) U.S. Cl. CPC A61H 15/02; A61H 2201/0192; A61F 7/10; A61F 7/02; A61H 2201/1647; A61H 2201/1695; A61H 2201/0214; A61H 2201/0207; A61H 2201/0228; A61H 2201/0242; A61H 2015/0021; A61H 2015/0035; A61H 2201/0257; A61F 2007/108; A61F 2007/0242; A61H 15/0092

(57) ABSTRACT

An improved therapeutic roller device comprising a cylindrical core and roller members originating from said cylindrical core. The roller members are separated, from each other, by a distance that is intended to generate lateral stretch on a muscle or other soft tissue. The amount of lateral stretch placed on a muscle, or other soft tissue, is proportional to the distance between roller elements up to a maximum distance at which lateral stretch can no longer be achieved. The diameter of the roller members determines the amount of longitudinal stretch placed on a muscle or other soft tissue. Additional longitudinal stretch is attained by ribbing of the roller members so that the roller members have projections. The length of the projections and distance between subsequent projections determine the extent of additional longitudinal stretch that can be placed on a muscle or other soft tissue.

### BACKGROUND OF THE INVENTION

### 1. Field of the Invention

The present invention relates in general to a therapeutic roller apparatus and pertains, more particularly, to an apparatus that provides enhanced therapeutic action for such applications, but not limited to, deep stimulation of skeletal muscles and soft tissues, self-massage, injury repair, and stretching.

### 2. Brief Discussion of the Related Art

At the present time, foam rollers exist. These rollers are used for therapeutic purposes such as for balance training, self-myofascial release, self-massage, and stretching. They are also used for golf swing practice, yoga, and Pilates. The rollers of this type are sold by, for example, Perform Better of Cranston, R.I. These may be identified as EVA Foam Rollers or PB Elite Molded Foam Rollers. These rollers may be formed in various lengths depending upon the particular application for the roller and may also be formed in different diameters.

The present invention is distinguishable from all devices in the prior art, including for example:
1) WO2015077643A3 by Norwood and Whittle which describes a myofascial release device that includes a rigid inner core having a first density surrounded by an outer layer having a second density. The second density may be less than the first density to promote myofascial release. The rigid inner core may be a hollow aluminum core and end caps may be placed on either end of the myofascial release device. The overall diameter of the myofascial release device may be between 1.5 and 3 inches, and preferably 2 inches. The inner core may be cooled during manufacturing such that, when the inner core returns to room temperature, the diameter of the inner core is greater than or equal to the inner diameter of the outer layer. The device by Norwood and Whittle is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
2) U.S. Pat. No. 9,132,055 to Wallace describes an athletic massage device with improved pressure absorption and distribution. The athletic massage device may comprise one or more massage rollers covered by one or more layers of pliant material, which allow a user improved control over how to adjust applied pressure. In some embodiments, devices are provided with a deep core construction with a pliant outer perimeter. A hard spine may house a motor and rechargeable battery to create vibrations, which may be adjustable. An inner core layer of a pliant material may overlay the spine. An outer surface layer of a more pliant material may overlay the inner core. Protrusions from the spine may extend into the inner core layer to improve pressure or energy transmission. Embodiments may be provided in the form of balls, massage sticks, rolling pins, or dumbbells. A docking station may provide a recharge connection and storage for the massage rollers. The device by Wallace is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
3) WO1992000053A1 by Teyssier describes an exercise device comprising a hollow rigid tube wrapped in a layer of structural foam. A vibrating sub-assembly is centrally housed in the tube, a source of electricity is housed at a first end of the tube, and a power supply control device is housed at the other end of the tube. The exercise device can be used as an axial back support unit for a user and provides spinal relaxation, care and rehabilitation. The device by Teyssier is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
4) US20100113992 to Godfrey and Warner describes a vibrational roller utilizing a motorized vibrational drive using a plurality of eccentric weights and associated supports. The weights are positioned along the axis of rotation in a manner to synchronize them and minimize vibrational dissipation throughout the roller. Each weight has at least one associated support. The supports then transmit vibrational forces to the surface of the roller in an evenly distributed manner. A second co-operational motor may be used to extend motor life and aid in initiating vibrations. A control panel and associated circuitry are utilized to alter vibrational characteristics. The roller may be battery powered or powered through a standard wall outlet. The roller itself is manufactured of a durable yet deformable material, such as foam rubber, and may be inserted into selectable sleeves of varying physical properties for desired effect. The device by Godfrey and Warner is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
5) US20080255477 to Woggon et al. describes a device that applies a vertically percussive vibration and non-symmetrical forces to a patient. The non-symmetrical forces may be generated by a variety of unevenly applied weights that are supported at appropriate body locations in a manner that upsets normal body load symmetry. The non-symmetrical forces in combination with vertically percussive vibrations induce neuromusculoskeletal proprioceptive re-education and development within a living body to correct musculoskeletal disorders including but not limited to scoliosis. A special posture modifying chair and head-orienting glasses may be used instead of or in addition to unevenly applied weights. The device by Woggon et al. is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
6) US20080070210 by Hitzmann describes a system made up of methods and techniques designed to improve the body's postural alignment, joint range, muscle hydration, bone density, and overall musculoskeletal health. The system combines a series of exercised designed to lengthen and strengthen muscles, fascial and myofascial tissue. The system is used in combination with a vibrating roller device, which can also be used with a head cradle. The application of the vibrational motion to the body in combination with the techniques described herein work to stimulate fascial tissue and confer benefits on the user which will improve the overall quality of life. The system and device by Hitzmann is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
7) US20080018167 by Fuji describes an omnidirectionally moving wheel that includes multiple rotating bodies and a wheel, wherein each rotating body has a flexibility to be able to bend a rotational axis, is bent to encircle an outer circumference of the wheel in a ring-like shape, and is compressed in a direction of the rotational axis of each rotating body to be disposed on the wheel in a manner to allow rotation centered around each rotating body's secured rotational axis extending along a plane perpendicular to the rotational axis of the wheel. The device by Fuji is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
8) US20040267173 to Mangano describes a battery operated flexible massage device that includes a ball-and-socket modular plastic spine and an electric vibrator unit, covered by a flexible foam tube. The tube containing the spine and the vibrator unit is covered with a padded fabric cylindrical shell. The ends of the shell are provided with zippers which allow access to battery compartments. According to the presently preferred embodiment, the vibrator unit is centrally located adjacent to the flexible plastic spine. Two battery compartments are provided, one at each end of the tube, each for containing two AA batteries. A control switch is mounted adjacent to one of the battery compartments. The device by Mangano is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
9) U.S. Pat. No. 7,285,101 to Tumey describes a vibrating transducer that includes a rigid housing; an electric motor enclosed within the rigid housing and having attached thereto an eccentric weight; and wherein the electric motor is supported within the rigid housing by a flexible motor mount. The rigid housing is preferably a cylindrically shaped tube. The flexible motor mount may be formed of a foam cushion wrapped substantially about the electric motor. A driver circuit, which may include may include a current amplifier or timing sub-circuits, may be provided for facilitating operation of the electric motor. The device by Tumey is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
10) U.S. Pat. No. 7,137,926 to Barrows describes a roller device in which the outside of an elongated exercise roller formed of a compressible, foam material, is modified. Instead of being cylindrical, a portion of the outside has a gentler curvature over part. That change makes balancing easier. The device by Barrows is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
11) U.S. Pat. No. 6,647,572 to Lee describes a cushion comprising a sponge having a predetermined length and a circular cylinder-shaped configuration; a plurality of vibrating motors embedded in the sponge; an inner envelope opened at one end thereof, for allowing the sponge to be inserted therein, with a predetermined space defined there between; cushioning segments filled into the space defined between the sponge and the inner envelope; a frame made of plastic material and fitted into the opened one end of the inner envelope to be brought at one end thereof into contact with the sponge, the frame having a cylindrical configuration; a rechargeable battery positioned in the frame and connected through electric wires to the vibrating motors; a frame cover fastened to the frame to close the other end of the frame; an aromatic case fixed to an inner surface of the frame cover to be positioned in the frame and receive therein an aromatic; an aromatic case cover attached to the frame cover and defined with a plurality of slots which are opened and closed by a plurality of sliding plates, respectively, so that an amount of the aromatic which is given out through the slots can be adjusted; and an outer envelope made of cloth and capable of being opened and closed at one end thereof, for enclosing the resultant combination. The device by Lee is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
12) U.S. Pat. No. 5,830,161 to Cosmano describes a foot massager built around a cylinder that preferably mounts two axially spaced longitudinally ribbed, bulging sleeves separated by a radially extended central partition disk and captured at their outer ends by roller disks, each having end stops to prevent the massager from rolling too far. The sleeve has alternating short and substantially compressible tall ribs to clutch the skin of a user's foot and thereby enhance the soothing effect. The cylinder mounts a vibrator internally. The user places his feet respectively on the two bulged sleeves, turns on the vibrator, and rolls his feet forward and backward in a reciprocating motion that rolls the vibrator on the floor beneath his feet. The configuration of the ribbed sleeves is such that the user can roll his feet against curved end ramps defined on both the inside (the side closer to the other foot) and outside of each foot for a complete massage of all foot surfaces. The device by Cosmano is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
13) U.S. Pat. No. 5,637,065 to Chang describes an exercise bar device comprising a first hollow bar, a second hollow bar, a massage sleeve extending proximally from the second hollow bar, a first foam tube enclosing the first hollow bar, a second foam tube enclosing the second hollow bar, a switch cover covering a distal end of the second hollow bar, a hexagonal rod inserted in the first hollow bar, a brake ring and a friction disk disposed on the hexagonal rod, a nut engaging with an outer thread of a threaded end of the hexagonal rod, a large number of protrusions disposed on an outer periphery of the massage sleeve, an inner sleeve disposed in the massage sleeve and connected to the second hollow bar, a spacing defined between the second hollow bar and the massage sleeve, a motor disposed in the inner sleeve, and a coiled spring and an elastic plate disposed in the spacing. The first hollow bar has an end disk. The end disk couples with the elastic plate. The rod is rotated to increase or decrease the torsion resistance between the hollow bars. The device by Chang is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
14) U.S. Pat. No. 5,554,102 to Chiou describes a portable massaging device comprising a cylindrical body in which a power unit is housed and over which a massaging set is fitted. The power unit is composed of a motor, a battery set, a cam and a conducting member. The massaging set is composed of a plurality of fitting members and massaging nipples. The fitting members are provided respectively with a plurality of receiving recesses in which the massaging nipples are held. The fitting members are further provided respectively in the connection ends thereof with a plurality of mortises and tenons, by way of which the fitting members are held together. An end cap is fastened to one end of the cylindrical body for locating the massaging set and for shielding the power unit. A handle is fastened at one end thereof with the end cap and at another end thereof with the conducting member. The device by Chiou is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
15) U.S. Pat. No. 4,136,867 to Wilkin describes an exercise wheel with a noncircular periphery preferably formed by a series of equiangularly spaced teeth. Handles are mounted on ends of an axle projecting through the wheel. As the wheel is moved along the floor, vibrations are imparted to the user through the axle and handles. The device by Wilkin is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
16) U.S. Pat. No. 3,970,078 to Rogers describes a massage roller having a broad rim with tapering sides in which are located alternate series of curved ridges and of rectangular based pyramids and pyramid peaks. A plane resting on two adjacent pyramid peaks would be somewhat spaced from the intermediate curved ridge. A roller handle frame is shaped somewhat like a capital letter D lying on its flat side. The curved upper side has four finger fitting recesses on its under side, providing a hand fitting handle. The bottom flat side provides an elongate chassis having an axle at each end extending on opposite sides, there thus being four rollers supported by the frame. Lightening grooves are provided along opposite sides of each elongate portion including the chassis, the curved handle and the connecting stanchions at each end between the handle and the chassis. The entire frame and rollers may be of rigid molded plastic for light weight, but also may be entirely of a suitable metal or other material. The handle is of a convenient size to be grasped by one hand to roll the massage rollers over fleshy and fat or “cellulite” areas of a human body to knead and manipulate the flesh and fat and thus break down the gel-like lumps of cellulite and dissipate them through normal body circulatory functions. In addition, it massages, tones and strengthens the body as it is rolled thereover, and is also used for this function alone. The device by Rogers is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
17) U.S. Pat. No. 2,939,455 to Clifford describes a machine known in the trade as a barrel roller, which is used for body massage, that has a horizontally mounted drum or barrel that carries around its periphery a multiplicity of long thin fluted rollers parallel to the axis of the drum and individually free to rotate. When the machine is in operation, the drum is rotated at a moderate speed while a person holds some portion of his body against the periphery of the drum, the ridges and grooves in the individual rollers as they revolve serving to massage the body. The device by Clifford is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.
18) U.S. Pat. No. 2,461,413 to Dirand describes an improved massaging roller. One of the objects thereof is to provide a simple, efficient, and inexpensive massaging roller for the purpose of effecting a better kneading action of the roller upon the tissues of the body over which it is used. The characteristics of the massage roller are such that the roller is made to have an eccentric movement or rolling action with respect to the shaft which constitutes its axis of rotation, such eccentric rolling action being spring cushioned. Another object is to provide a massaging roller characterized by the fact that it is normally mounted concentrically upon an operating shaft which carries two handles for rolling it at opposite ends thereof. The massaging roller, however, is journaled upon the operating shaft through intervention of circular resilient bodies which act as yieldable cushions. A further object of the invention is to provide a novel form of massaging roller which may not only be rolled upon the part to be massaged but pressed there against as well. The device by Dirand is distinguishable from the present invention in that the present invention has a core from which roller members originate, with spaces between the roller members for lateral stretch of skeletal muscle, or other tissue, and ribbing of the roller members to increase the longitudinal stretch of skeletal muscle and other tissue.

### SUMMARY OF THE INVENTION

It is an object of the present invention to improve the application of these rollers by incorporating roller members with spaces between them to attain lateral stretching of skeletal muscles and other soft tissues. It is a further object of the present invention to improve the application of these rollers by incorporating ribs to increase the longitudinal stretch of skeletal muscles and other soft tissue and to increase the depth of stimulation. Thus, in accordance with one aspect of the present invention, there is provided a therapeutic roller apparatus that comprises a cylindrical core and roller members originating from said cylindrical core. Each roller member is separated from the next one by a space, the distance of which is adjustable to produce a desired amount of stretch to a particular skeletal muscle, a group of skeletal muscles, or other soft tissue. For example, relatively wider spaces would be required to produce lateral stretch of the large muscles of the thigh whereas relatively smaller spaces would be sufficient to stretch the gastrocnemius muscle laterally. The roller members are sufficiently rigid to maintain their form with use over time, but soft enough to be used on bare skin. Further, in accordance with another aspect of the present invention is provided roller members that are removable. And, even further, in accordance with another aspect of the present invention are roller members that provide a thermal stimulus by functioning either as heat packs or ice packs. Unlike existing rollers that provide only a longitudinal stretch of skeletal muscle or other soft tissue, the present invention provides, simultaneously, both a longitudinal and lateral stretch, resulting in a deeper stimulation of the skeletal muscle or other soft tissue.

### BRIEF DESCRIPTION OF THE DRAWINGS

It should be understood that the drawings are provided for the purpose of illustration only and are not intended to define the limits of the disclosure. The foregoing and other objects and advantages of the embodiments described herein will become apparent with reference to the following detailed description when taken in conjunction with the accompanying drawings in which:

FIG. 1 is a view of the therapeutic roller device showing the cylindrical core and roller members;

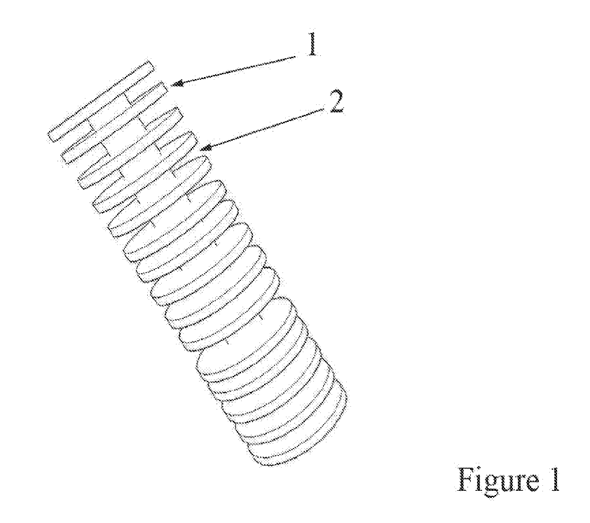

FIG. 2 is an alternate view of the therapeutic roller device showing the cylindrical core and roller members;

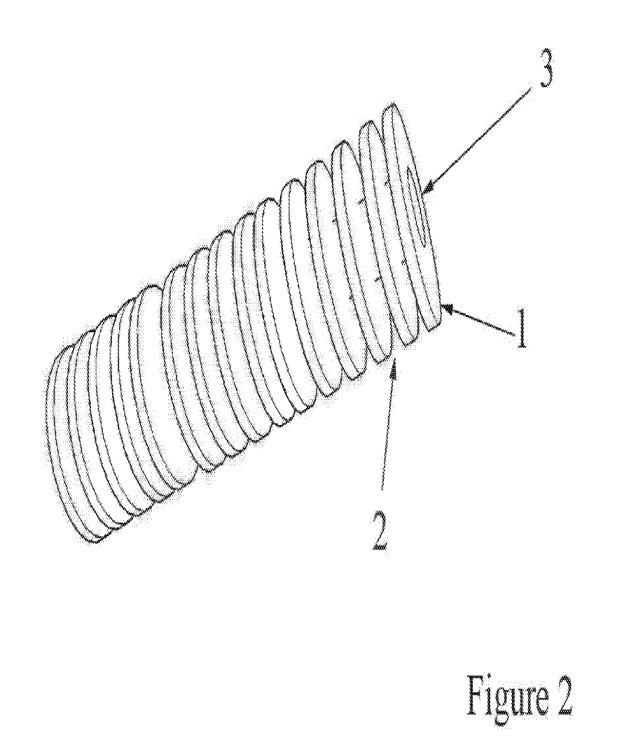

FIG. 3 is a view of the therapeutic roller device in which some of the roller members are ribbed;

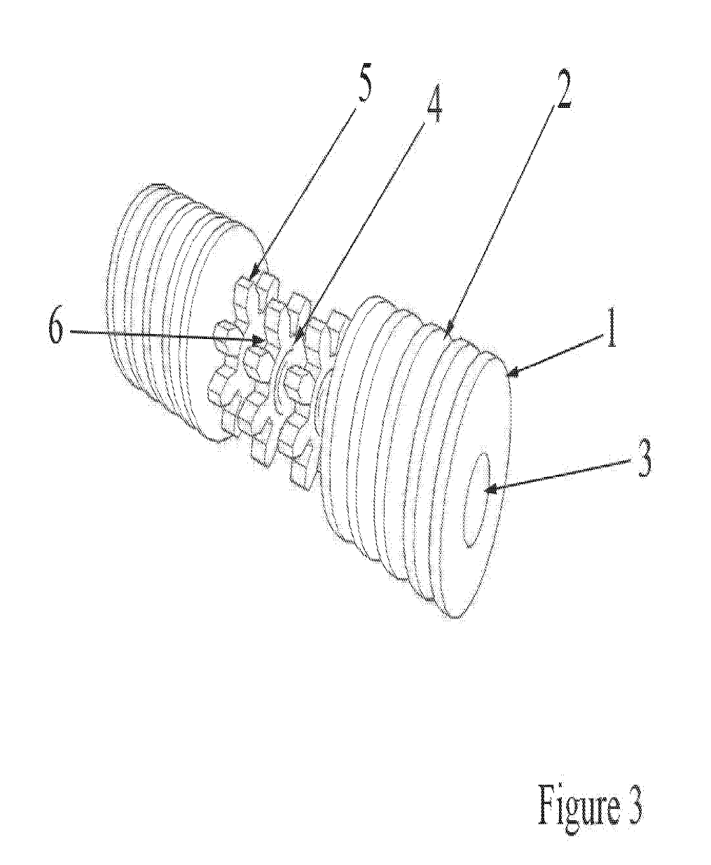

FIG. 4 is an alternate view of the therapeutic roller device in which some of the roller members are ribbed;

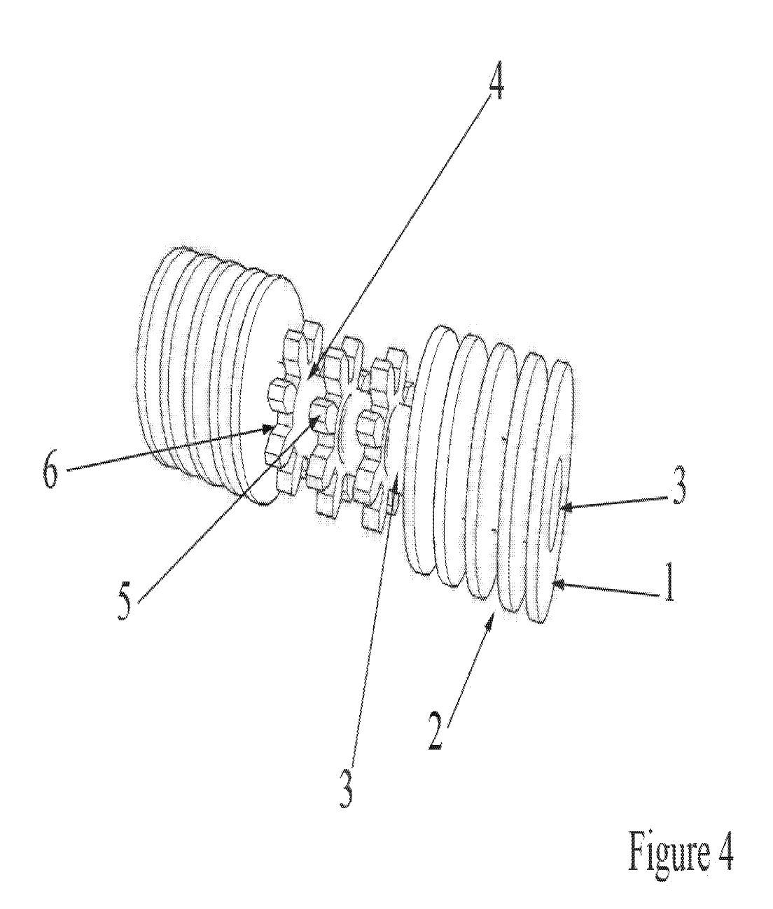

FIG. 5 is an alternative view of the therapeutic roller device in which some of the roller members are ribbed; and

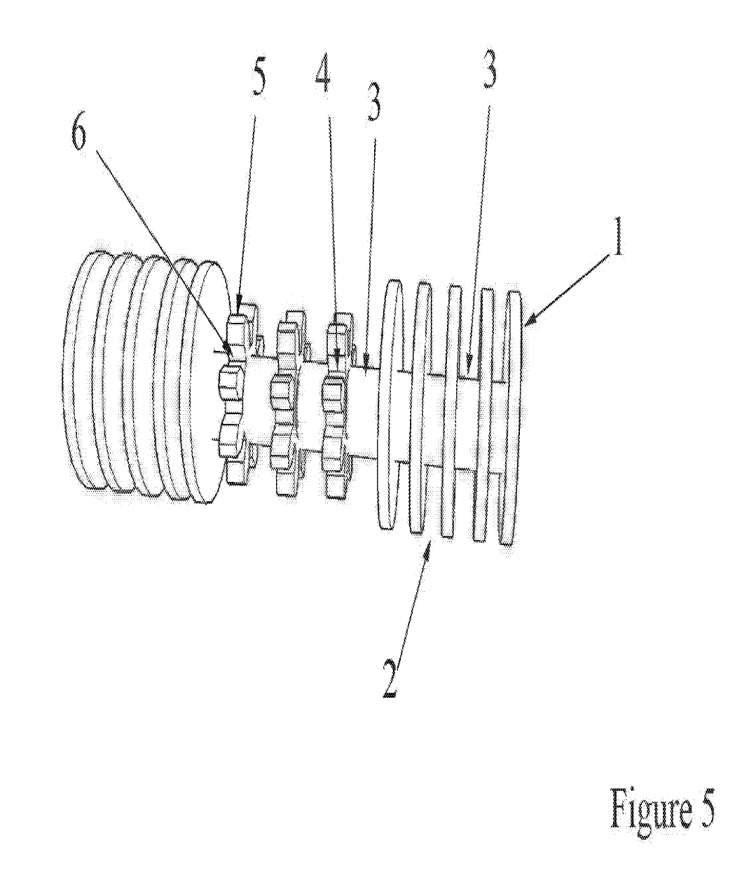

FIG. 6 is a view of the therapeutic roller device in which all of the roller members are ribbed.

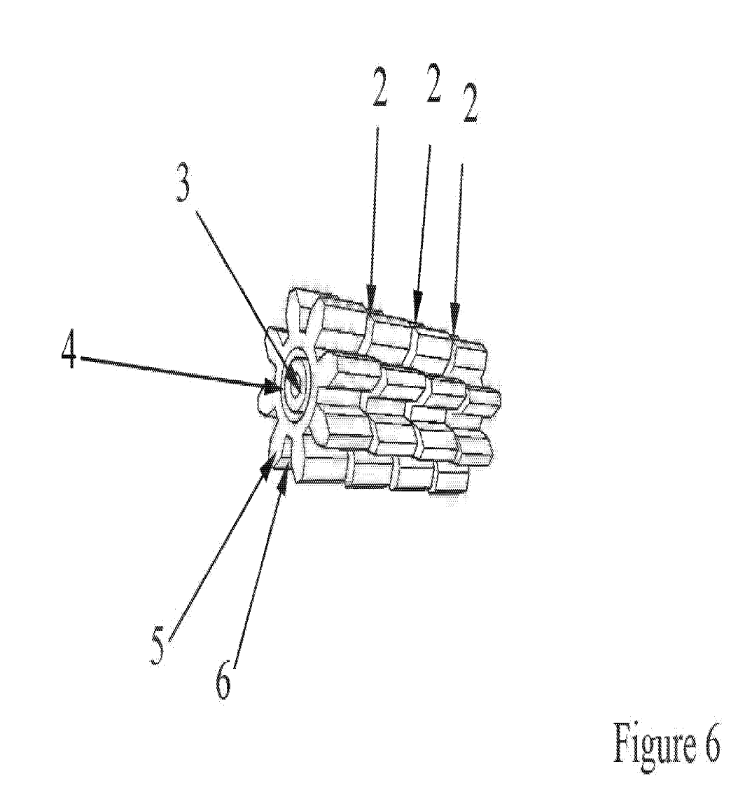

The figures accompanying this document, as well as the detailed description of embodiments, are exemplary only, and are not intended to show the bounds of the current invention. Also, the figures are not to scale.

### DETAILED DESCRIPTION OF EXEMPLARY EMBODIMENTS OF THE INVENTION

In accordance with one aspect of the present invention is an improved therapeutic roller device comprising a cylindrical core (3) from which projects roller members (1). The roller members are separated from each other by a gap (2). The diameter of the roller members (1), which is typically in the range of about 2 inches to about 20 inches, is directly proportional to the amount of longitudinal stretch that the therapeutic roller device can achieve on skeletal muscles or other soft tissue. The gap (2) between the roller members is adjustable and directly proportional to the amount of lateral stretch. The gap (2) is typically in the range of about half an inch to about three inches, depending on the size of the target skeletal muscle or other soft tissue. In a preferred embodiment, the improved therapeutic roller device is a single molded construct that is rigid enough to maintain its form with use over time, but not so hard that it cannot be used on bare skin, and in another preferred embodiment the roller members are removable.

In another preferred embodiment is an improved therapeutic roller device comprising a cylindrical core (3) from which projects roller members (1), some of said roller members ribbed (4) to form projections (5) that increase the amount of longitudinal stretch and the depth of tissue stimulation. Adjacent projections (5) are separated by troughs (6), which are typically in the range of about 0.25 inch to about 3 inches. The wider the trough, the greater the extent of longitudinal stretch of skeletal muscle and the greater the depth of tissue stimulation. Also, the longer the projections (5) the greater the longitudinal stretch of skeletal muscle and the depth of tissue stimulation.

In yet another preferred embodiment is an improved therapeutic roller device comprising a cylindrical core (3) from which projects ribbed roller members (4). The projections (5) of the ribbed roller members are separated by troughs (6), the width of which determines the extent of longitudinal stretch of skeletal muscle. The greater the width, which is typically in the range of about 0.25 inch to about 3 inches, the greater the longitudinal stretch on skeletal muscles and the greater the depth of tissue stimulation. The ribbed roller members (4) are separated from each other by gaps (s) that are directly proportional to magnitude of lateral stretch of skeletal muscles and other soft tissues. The gaps (2) range from about 0 to about 3 inches. The dimensions are for a standard adult weighing from about 100 lbs to about 200 lbs and from about 4 feet to about six feet in height, and would be adjusted up or down to accommodate for larger or smaller sizes.

In a preferred embodiment, the present invention would be made of rubber or plastic, but could be made of any material with sufficient rigidity, to maintain its form with use over time, without being too punishing on bare skin. In other words, any material is good for manufacture if it is not damaging to the skin, with normal use, and maintains its shape, over time, with normal use.

In the most preferred embodiment, the roller members comprise heat packs or cold packs, said roller members removable, for example, to place in a freezer or microwave oven.

Patent Citations

| | | Publication | | |
|---|---|---|---|---|
| Cited Patent | Filing date | date | Applicant | Title |
| U.S. Pat. No. 2,461,413 | Jul. 18, 1946 | Feb. 8, 1949 | Irwin F Mills | Massaging roller |
| U.S. Pat. No. 2,939,455 | Dec. 19, 1957 | Jun. 7, 1960 | Arnold Clifford R | Barrel roller massage machine |
| U.S. Pat. No. 3,970,078 | Mar. 10, 1975 | Jul. 20, 1976 | Rogers Jr Charles G | Hand roller massage device |
| U.S. Pat. No. 4,136,867 | May 31, 1977 | Jan. 30, 1979 | Wilkin Douglas G | Vibrating exercising wheel |
| U.S. Pat. No. 5,554,102 | Apr. 20, 1995 | Sep. 10, 1996 | Chiou; Shih-Kuen | Portable massaging device |
| U.S. Pat. No. 5,637,065 | Jul. 25, 1996 | Jun. 10, 1997 | Chang; Sreter | Massage exercise bar device |
| U.S. Pat. No. 5,830,161 | Oct. 21, 1996 | Nov. 3, 1998 | Cosmano; Robert J. | Alternating ribbed foot massager |
| U.S. Pat. No. 6,647,572 | Aug. 6, 2002 | Nov. 18, 2003 | Kwang-Ho Lee | Cushion having embedded therein |
| | | | | vibrating motors |
| U.S. Pat. No. 7,137,926 | Mar. 16, 2004 | Nov. 21, 2006 | Stacy Barrows | Foam roller |
| U.S. Pat. No. 7,285,101 | May 26, 2005 | Oct. 23, 2007 | Solutions For | Vibrating transducer with provision |
| | | | Thought, Llc | for easily differentiated multiple |
| | | | | tactile stimulations |
| US20040267173 | Jun. 26, 2003 | Dec. 30, 2004 | Joy Mangano | Battery operated flexible massage tube |
| US20080018167 | Dec. 13, 2005 | Jan. 24, 2008 | Shinichiro Fuji | Omnidirectionally Moving Wheel, |
| | | | | Moving Device, Carrying Device, |
| | | | | and Massage Device |
| US20080070210 | Mar. 26, 2007 | Mar. 20, 2008 | Sue Hitzmann | System and methods for promoting health |
| US20080255477 | Jun. 20, 2008 | Oct. 16, 2008 | Woggon Dennis A | Method of Neuromusculoskeletal |
| | | | | Proprioceptive Re-education and |
| | | | | Development of a Living Body and |
| | | | | Apparatus Therefor |
| US20100113992 | Nov. 6, 2009 | May 6, 2010 | Brian P. Godfrey | Vibrating Massage Roller Utilizing a |
| | | | | Plurality of Supports and Eccentric |
| | | | | Weights |
| WO1992000053A1 | Jun. 25, 1991 | Jan. 9, 1992 | Jean Teyssier | Backbone exercise device |
| U.S. Pat. No. 9,132,055 | Sep. 17, 2013 | Sep. 15, 2015 | Carey M Wallace | Athletic massage device |
| WO2015077643A3 | Nov. 21, 2014 | Nov. 12, 2015 | Mfr Products, Inc. | Therapeutic device to assist in |
| | | | | myofascial release, and method of use |

| Classifications | |
|---|---|
| U.S. Classification | 601/46 |
| International Classification | A61H1/00 |
| Cooperative Classification | A61H15/00, A61H2015/0014, A61H23/0263, A61H2201/1685, |
| | A61H15/0092 |

That which is claimed is:

1. A therapeutic roller device for myofascial release, pain management, and the treatment of injuries, comprising a cylindrical core and roller members originating from said cylindrical core.

2. The device of claim 1 wherein the roller members are adjustable, such that the spaces between said roller members can be altered to fit the ideal dimensions for each individual user and adjustable for any particular muscle or other soft tissue for which it is being used.

3. The device of claim 1 wherein the roller members are ribbed to enhance longitudinal soft tissue stretch.

4. The device of claim 3 wherein the roller members are adjustable and removable, such that the spaces between said roller members can be altered to fit the ideal dimensions for each individual user and adjustable for any particular muscle or other soft tissue for which it is being used.

5. The device of claim 2 wherein the roller members are removable from the core.

6. The device of claim 4 wherein the roller members comprise of ice packs that can be removed and chilled in a freezer until ready for use to provide myofascial release while reducing inflammation for a more efficient pain management and faster recovery from injury.

7. The device of claim 5 wherein the roller members comprise of ice packs that can be removed and chilled in a freezer until ready for use to provide myofascial release while reducing inflammation for a more efficient pain management and faster recovery from injury.

8. The device of claim 1 wherein the roller members comprise of ice packs that can be removed and chilled in a freezer until ready to use to provide myofascial release while reducing inflammation for a more efficient pain management and faster recovery from injury.

9. The device of claim 2 wherein the roller members comprise of ice packs and wherein the entire device can be chilled in a freezer until ready to use to provide myofascial release while reducing inflammation for a more efficient pain management and faster recovery from injury.

10. The device of claim 3 wherein the roller members comprise of ice packs and wherein the entire device can be chilled in a freezer until ready to use to provide myofascial release while reducing inflammation for a more efficient pain management and faster recovery from injury.

11. The device of claim 1 wherein the roller members comprise of heat packs and wherein the entire device can be placed in a heat source, preferably microwave oven or boiling water, such that, upon using, the device provides myofascial release while increasing circulation for a more efficient pain management and faster recovery from injury.

12. The device of claim 2 wherein the roller members comprise of heat packs and wherein the entire device can be placed in a heat source, preferably microwave oven or boiling water, such that, upon using, the device provides myofascial release while increasing circulation for a more efficient pain management and faster recovery from injury.

13. The device of claim 3 wherein the roller members comprise of heat packs and wherein the entire device can be placed in a heat source, preferably microwave oven or boiling water, such that, upon using, the device provides myofascial release while increasing circulation for a more efficient pain management and faster recovery from injury.

14. The device of claim 4 wherein the roller members comprise of heat packs and wherein said roller members are removable from the cylindrical core and can be placed in a heat source, preferably microwave oven or boiling water, such that, upon using, the device provides myofascial release while increasing the circulation for a more efficient pain management and faster recovery from injury.

15. The device of claim 5 wherein the roller members comprise of heat packs and wherein said roller members are removable from the cylindrical core and can be placed in a heat source, preferably microwave oven or boiling water, such that, upon using, the device provides myofascial release while increasing the circulation for a more efficient pain management and faster recovery from injury.

16. The device of claim 1 wherein the roller members comprise of electrical heating elements, and wherein the amount of heat released by the device is controllable by the user.

17. The device of claim 2 wherein the roller members comprise of electrical heating elements, and wherein the amount of heat released by the device is controllable by the user.

18. The device of claim 3 wherein the roller members comprise of electrical heating elements, and wherein the amount of heat released by the device is controllable by the user.

19. The device of claim 1 wherein the roller members are self-chilling, and wherein the roller members comprise of a refrigerant under pressure connected, in a closed system, to a refrigerant escape reservoir in the cylindrical core, and wherein chilling is achieved by opening a valve to rapidly release refrigerant from the roller members into the reservoir and wherein the process is repeatable until equilibrium is achieved and wherein said equilibrium is reversed by pumping refrigerant from the reservoir in the cylindrical core back into the roller members.

20. The device of claim 2 wherein the roller members are self-chilling, and wherein the roller members comprise of a refrigerant under pressure connected, in a closed system, to a refrigerant escape reservoir in the cylindrical core, and wherein chilling is achieved by opening a valve to rapidly release refrigerant from the roller members into the reservoir and wherein the process is repeatable until equilibrium is achieved and wherein said equilibrium is reversed by pumping refrigerant from the reservoir in the cylindrical core back into the roller members.
